(19) United States
(12) Patent Application Publication
(10) Pub. No.: US 2024/0024522 A1
(43) Pub. Date: Jan. 25, 2024

(54) HEXAHEDRAL HEATING HIGH-TEMPERATURE STERILIZATION BIOLOGICAL SHAKING TABLE

(71) Applicant: SHANGHAI ZHICHU INSTRUMENT CO., LTD., Shanghai (CN)

(72) Inventors: Kai CAO, Shanghai (CN); Bing QIAN, Shanghai (CN); Jun QIAN, Shanghai (CN)

(21) Appl. No.: 18/156,299

(22) Filed: Jan. 18, 2023

(30) Foreign Application Priority Data
Jul. 20, 2022 (CN) 202210853170.9

(51) Int. Cl. A61L 2/04; B01L 7/00; A61L 2/26

(52) U.S. Cl. CPC A61L 2/04; B01L 7/00; A61L 2/26; B01L 2300/1827; B01L 2400/0433; A61L 2202/122; A61L 2202/121

(57) ABSTRACT

The invention discloses a six-face heating high-temperature sterilization biological shaking table, comprises a box body and a transmission mechanism, one side of the box body is provided with an opening, the opening is provided with a door body, the box body is internally provided with an inner container, and the side surface of the inner container is composed of five-surface concentric-square-shaped high-temperature-resistant heating wires; the two rocking shafts are rotationally arranged in the box body, a supporting plate is rotationally arranged between the ends of the two rocking shafts, the other end of the belt is connected with the outer wall of the belt pulley in a winding mode, the end of the belt pulley is rotationally inserted into the inner side wall of the first mounting groove through a pin shaft, and the end of the pin shaft of the belt pulley rotationally extends into the box body.

### BACKGROUND OF THE INVENTION

### 1. Field of the Invention

The invention relates to the technical field of molecular biology, and more particularly, to a six-face heating high-temperature sterilization biological shaking table.

### 2. Description of the Related Art

A heating high-temperature sterilization biological shaking table is a laboratory equipment used for sterilizing biological materials through high-temperature heating and shaking. This type of sterilization is also known as “dry heat sterilization.” The equipment typically has a heating chamber that is able to reach high temperatures, such as 160-180° C. Inside the chamber, there is a platform or table that can shake or agitate the biological materials, such as petri dishes or test tubes, to ensure that they are exposed to the heat evenly. The high-temperature heating process kills or inactivates microorganisms, bacteria, and other contaminants that may be present in the biological materials, thus making them safe for further experimentation or use. The shaking table helps to ensure that the heat is distributed evenly and that all surfaces of the materials are exposed to the heat for the same amount of time. This type of sterilization is effective for killing microorganisms that are not killed by lower temperatures, such as thermophiles and endospores. It's used in research lab, food industry, medical or pharmaceutical industry and other places where sterilization is required.

In the field of existing biological shaking tables, an interior of a box body of the shaking table is mainly sterilized with ultraviolet rays, or by spraying alcohol or hydrogen peroxide. However, those methods have a disadvantage that corners are not completely sterilized, which affects the final cultivation effects; in addition, the box body has poor thermal insulation performance and is prone to generation of condensed water, it is easy to cause bacterial infection and affect the actual cultivation. Moreover, an internal transmission mechanisms, a motor, a fan and other accessories cannot withstand high temperature, so they are easy to damage and product performance will be affected. With those considerations, a six-face heating high-temperature sterilization biological shaking table is designed herein for solving the above-mentioned problems.

### SUMMARY OF THE INVENTION

An object of the present invention is to provide a six-face heating high-temperature sterilization biological shaking table, so as to solve the problems mentioned above

In order to achieve the above-mentioned object, the present invention provides the following technical solution: a six-face heating high-temperature sterilization biological shaking table, comprising: a box body and a transmission mechanism, one side of the box body is provided with an opening, the opening is provided with a door body, the door body functions as a component for closing and opening the box body, so it is easy to access parts to be sterilized.

The box body is internally provided with an inner container, and the side surface of the inner container is composed of five-face concentric-square-shaped high-temperature-resistant heating wires, that is, a heating system is formed. Five faces of the inner container are composed of specially designed high-temperature-resistant heating wires evenly distributed inside the inner container, to ensure that heat is evenly distributed at an interior of the box body, so that heat can be distributed anywhere in the box body to facilitate high-temperature rapid sterilization operations.

The transmission mechanism comprises a motor, a belt pulley and two rocking shafts, wherein the two rocking shafts are rotationally arranged in the box body, a supporting plate is rotationally arranged between the ends of the two rocking shafts. The supporting plate functions as a support for carrying the parts to be sterilized. The two rocking shafts rotate inside the box body, which can drive the supporting plate to make a swinging motion synchronously, so that the parts to be sterilized can be subjected to high-temperature sterilization operations in all directions. In this way, high-efficiency sterilization can be achieved, time required for the sterilization process can be shortened, and the efficiency of sterilization is improved.

A first mounting groove is formed at a side wall of the box body, the motor is fixedly arranged inside the first mounting groove, a belt is connected with an end of a power shaft of the motor in a winding mode, the other end of the belt is connected with an outer wall of the belt pulley in a winding mode, the end of the belt pulley is rotationally inserted into an inner side wall of the first mounting groove through a pin shaft, and the end of the pin shaft of the belt pulley rotationally extends into the box body and is fixedly connected with the end of one rocking shaft. Power provided by the motor drives the belt to rotate between the power shaft and the belt pulley, the belt pulley can drive the pin shaft at the end to rotate synchronously, that is, the pin shaft can drive one of the rocking shafts to rotate. The supporting plate is also driven to rotate synchronously, so that the two rocking shafts can rotate synchronously, and the supporting plate performs a circular swing operation, thus, sterilization efficiency for the parts to be sterilized is improved.

In a further embodiment, a thermal-insulating layer is arranged between an interior of the box body and the inner container, and the thermal-insulating layer is made of a high-temperature-resistant thermal-insulating material.

In a further embodiment, the door body is composed of glass and door liner, the door body is filled with the high-temperature-resistant thermal-insulating material.

In a further embodiment, the heating wires of the inner container are wrapped in aluminum foil.

In a further embodiment, a second mounting groove is formed at an outer wall of the box body, the second mounting groove is internally provided with a circulating fan, an inner wall of the second mounting groove is provided with an air inlet in communication with the interior of the box body. The circulating fan is used to provide wind. Wind blows into the interior of the box body through the air inlet, then circulates inside the box body, so as to increase the flow rate of high-temperature air, and to improve the sterilization efficiency.

In a further embodiment, an evaporator is embedded into the side wall of the box body, the inner wall of the box body is provided with an air outlet in communication with ends of the evaporator. The evaporator serves to evaporate air entering into and exiting from the evaporator through the air outlet. The air from the air outlet cooperates with the air from the air inlet to form a circulating wind, which further improves the sterilization efficiency.

Compared with the prior art, the present invention has the following beneficial effects that the invention is a six-face heating high-temperature sterilization biological shaking table, high-temperature sterilization is used for the sterilization of the biological shaking table, so that a complete sterilization can be ensured. In the meantime, since a six-face heating system is used in the interior of the box body, generation of condensed water is effectively avoided, stable and uniform internal temperature is ensured. In addition, the transmission mechanism, the motor, the circulating fan, and other accessories can be arranged outside the box body, so they can be better protected, and can be avoided from being damaged due to the application of high temperature and from affecting the product performance.

### BRIEF DESCRIPTION OF THE DRAWINGS

FIGS. 1, 2 and 3 are sectional views of a main structure of the present invention; and

FIG. 4 is a schematic view showing a structure of an inner container of the present invention.

The following call out list of elements can be a useful guide for referencing the element numbers of the drawings.
1 box body
3 Transmission mechanism
12 door body
2 inner container
31 motor
32 belt pulley
34 rocking shafts
33 belt
4 circulating fan
5 evaporator
11 thermal-insulating layer

### DETAILED DESCRIPTION

The present invention will be described clearly and fully hereinafter with reference to technical solutions in embodiments of the invention. It is apparent that the embodiments illustrated herein represent only a part of embodiments instead of all the embodiments. Other embodiments obtained from the embodiments presented herein by those ordinary skilled in the art without any creative work are all construed to fall into the scope of the invention.

### Example 1

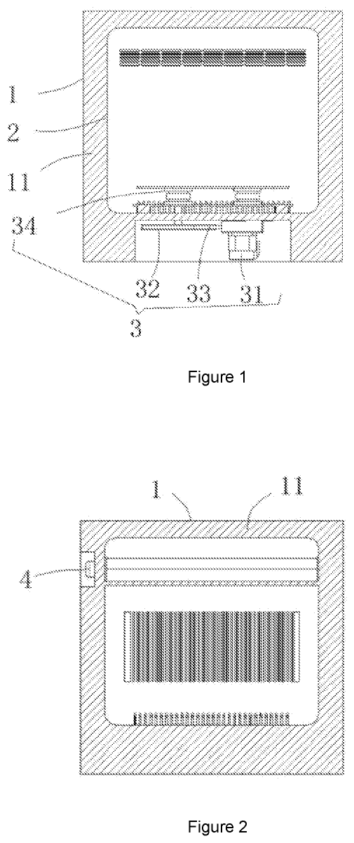

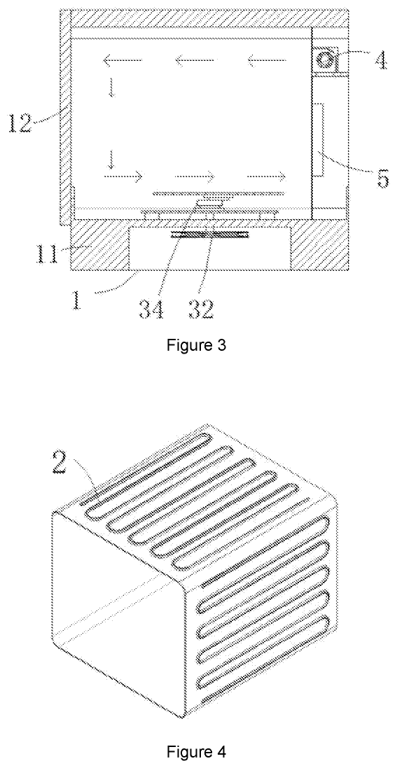

Please referring to FIGS. 1-4, in this embodiment, a six-face heating high-temperature sterilization biological shaking table is provided, comprising: a box body 1 and a transmission mechanism 3, one side of the box body 1 is provided with an opening, the opening is provided with a door body 12, the door body 12 is a component for closing and opening the box body 1, so it is easy to access parts to be sterilized.

The box body 1 is internally provided with an inner container 2, and the side surface of the inner container 2 is composed of five-face concentric-square-shaped high-temperature-resistant heating wires, that is, a heating system is formed. Five faces of the inner container 2 are composed of specially designed high-temperature-resistant heating wires evenly distributed inside the inner container 2, to ensure that heat is evenly distributed at an interior of the box body 1, so that heat can be distributed anywhere in the box body 1 to facilitate high-temperature rapid sterilization operations.

In addition, heating on each side of the inner container 2 is controlled in an independent and intelligent manner, to ensure a stable and uniform temperature on each side of the inner container 2.

The transmission mechanism 3 comprises a motor 31, a belt pulley 32 and two rocking shafts 34, wherein the two rocking shafts 34 are rotationally arranged in the box body 1, a supporting plate is rotationally arranged between the ends of the two rocking shafts 34. The supporting plate functions as a support for carrying the parts to be sterilized. The two rocking shafts 34 rotate inside the box body 1, which can drive the supporting plate to make a swinging motion synchronously, so that the parts to be sterilized can be subjected to high-temperature sterilization operations in all directions. In this way, high-efficiency sterilization can be achieved, and time required for the sterilization process can be shortened.

The two rocking shafts 34 are customized with a special process to ensure a normal operation in a high-temperature environment.

A first mounting groove is formed at a side wall of the box body 1, the motor 31 is fixedly arranged inside the first mounting groove, a belt 33 is connected with an end of a power shaft of the motor 31 in a winding mode, the other end of the belt 33 is connected with an outer wall of the belt pulley 32 in a winding mode, the end of the belt pulley 32 is rotationally inserted into an inner side wall of the first mounting groove through a pin shaft, and the end of the pin shaft of the belt pulley 32 rotationally extends into the box body 1 and is fixedly connected with the end of one rocking shaft 34. Power provided by the motor 31 drives the belt 33 to rotate between the power shaft and the belt pulley 32, the belt pulley 32 can drive the pin shaft at the end to rotate synchronously, that is, the pin shaft can drive one of the rocking shafts 34 to rotate. The supporting plate is also driven to rotate synchronously, so that the two rocking shafts 34 can rotate synchronously, and the supporting plate perform a circular swing operation, so that the parts to be sterilized can fully contact the high-temperature air, thus, sterilization efficiency for the parts to be sterilized is improved.

The motor 31 and the belt 33 are completely arranged outside of the box body 1, so the motor 31 and the belt 33 are protected from the influence of high temperature, so as to protect the motor 31 and the belt 33, and influence of temperature rise of the motor 31 on the interior of the box body 1 can also be solved.

A second mounting groove is formed at an outer wall of the box body 1, the second mounting groove is internally provided with a circulating fan 4, an inner wall of the second mounting groove is provided with an air inlet in communication with the interior of the box body 1. The circulating fan 4 is used to provide wind. Wind blows into the interior of the box body 1 through the air inlet, then circulates inside the box body 1, so as to increase the flow rate of high-temperature air, and to improve the sterilization efficiency.

Moreover, the circulating fan 4 is arranged outside the box body 1, completely isolated from a high-temperature environment inside the box body 1. Therefore, it ensures that the circulating fan 4 can normally operate even in the high-temperature environment.

In the meantime, an evaporator 5 is embedded into the side wall of the box body 1, the inner wall of the box body 1 is provided with an air outlet in communication with ends of the evaporator 5. The evaporator 5 serves to evaporate air entering into and exiting from the evaporator 5 through the air outlet. The air from the air outlet cooperates with the air from the air inlet to form a circulating wind, which further improves the sterilization efficiency.

The use of high-temperature sterilization ensures a more complete sterilization. The box body 1 is designed in a way that generation of condensed water can be avoided. This new six-face heating structure allows the heating process to be carried out in a more stable and uniform way. In this way, problems in the prior art are effectively solved, product performance is improved, and different requirements in the actual use can be satisfied.

The six-face heating high-temperature sterilization biological shaking table solve problems found in the traditional sterilization method, that is, the sterilization is not implemented completely and it is easy to cause bacterial infection. The traditional box body 1 has a structure in which condensed water is easy to produce. In addition, for the traditional heating method, large temperature fluctuation occurs, and the traditional thermal insulation structure has a problem of lower thermal insulation efficiency.

### Example 2

With reference to FIGS. 1-4 again, improvements are made on the basis of Example 1:

the heating wires of the inner container 2 are wrapped in aluminum foil, and it is fully attached to the inner container 2, in order to ensure its heat can be uniformly conveyed to the interior of the box body 1.

A thermal-insulating layer 11 is arranged between an interior of the box body 1 and the inner container 2, and the thermal-insulating layer 11 is made of a high-temperature-resistant thermal-insulating material. The high-temperature-resistant thermal-insulating material is used between the box body 1 and the inner container 2 for ensuring heat will not transferred to an exterior of the box body 1 while carrying out the high-temperature sterilization, and for ensuring condensed water will not easily produced inside the box body 1.

In addition, the door body 12 is composed of glass and door liner, the door body 12 is filled with the high-temperature-resistant thermal-insulating material. As a result, stability of temperature around a door frame can be ensured, and generation of condensed water is avoided.

Embodiments of the present invention are illustrated herein, but those ordinary skilled in the art should be able to realize that the many modifications, variations and alternations can be made without departing from the principle and spirit of the present invention, and the scope of the invention shall be defined by the appended claims and their equivalents.

What is claimed is:

1. A six-face heating high-temperature sterilization biological shaking table, comprising:
a box body (1) and a transmission mechanism (3), one side of the box body (1) is provided with an opening, the opening is provided with a door body (12), the box body (1) is internally provided with an inner container (2), and the side surface of the inner container (2) is composed of five-face concentric-square-shaped high-temperature-resistant heating wires;
the transmission mechanism (3) comprises a motor (31), a belt pulley (32) and two rocking shafts (34), wherein the two rocking shafts (34) are rotationally arranged in the box body (1), a supporting plate is rotationally arranged between the ends of the two rocking shafts (34);
a first mounting groove is formed at a side wall of the box body (1), the motor (31) is fixedly arranged inside the first mounting groove, a belt (33) is connected with an end of a power shaft of the motor (31) in a winding mode, the other end of the belt (33) is connected with an outer wall of the belt pulley (32) in a winding mode, the end of the belt pulley (32) is rotationally inserted into an inner side wall of the first mounting groove through a pin shaft, and the end of the pin shaft of the belt pulley (32) rotationally extends into the box body (1) and is fixedly connected with the end of one rocking shaft (34).

2. The six-face heating high-temperature sterilization biological shaking table of claim 1, wherein a thermal-insulating layer (11) is arranged between an interior of the box body (1) and the inner container (2), and the thermal-insulating layer (11) is made of a high-temperature-resistant thermal-insulating material.

3. The six-face heating high-temperature sterilization biological shaking table of claim 1, wherein the door body (12) is composed of glass and door liner, the door body (12) is filled with the high-temperature-resistant thermal-insulating material.

4. The six-face heating high-temperature sterilization biological shaking table of claim 1, wherein the heating wires of the inner container (2) are wrapped in aluminum foil.

5. The six-face heating high-temperature sterilization biological shaking table of claim 1, wherein a second mounting groove is formed at an outer wall of the box body (1), the second mounting groove is internally provided with a circulating fan (4), an inner wall of the second mounting groove is provided with an air inlet in communication with the interior of the box body (1).

6. The six-face heating high-temperature sterilization biological shaking table of claim 5, wherein an evaporator (5) is embedded into the side wall of the box body (1), the inner wall of the box body (1) is provided with an air outlet in communication with ends of the evaporator (5).
